(19) United States
(12) Patent Application Publication
(10) Pub. No.: US 2020/0008606 A1
(43) Pub. Date: Jan. 9, 2020

(54) MARIJUANA BEVERAGE SPIKER AND METHOD

(71) Applicant: Adam Franklin Dickie, Mission (CA)

(72) Inventor: Adam Franklin Dickie, Mission (CA)

(21) Appl. No.: 16/411,531

(22) Filed: May 14, 2019

Related U.S. Application Data
(63) Continuation-in-part of application No. 16/029,518, filed on Jul. 6, 2018.

(51) Int. Cl. A47J 31/20; A23L 2/52; A23L 33/105; A23L 2/02; A61K 9/00; A61K 31/352

(52) U.S. Cl. CPC A47J 31/20; A23L 2/52; A23L 33/105; A23V 2002/00; A61K 9/009; A61K 9/0004; A61K 31/352; A23L 2/02

(57) ABSTRACT

A mesh bag filled with non-decarboxylated marijuana, decarboxylated marijuana and/or marijuana concentrates designed to fit in virtually any beverage container. Once submerged the liquid beverage absorbs the cannabinoids (THC/CBDs) and thereby become psycho-active. The Marijuana Beverage spiker is a food grade mesh bag filled with non-decarboxylated marijuana, decarboxylated marijuana and/or marijuana concentrates. The mesh bag is designed to fit into virtually any pre-existing beverage container and be added to the container by the consumer after purchase. Once submerged in liquid, the marijuana and/or marijuana concentrates in the MBS absorbs into the liquid beverage. The liquid beverage will absorb the cannabinoids (THC/CBDs) and become psycho-active.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of beverage solutions of existing art and more specifically relates to a marijuana beverage spiker and method.

### RELATED ART

Cannabis, also known as marijuana is a psychoactive drug from the Cannabis plant intended for medical or recreational use. The main psychoactive part of cannabis is tetrahydrocannabinol (THC), one of 483 known compounds in the plant, including at least 65 other cannabinoids. Cannabis can be used by smoking, vaporizing, within food, or as an extract. Cannabis is often used for its mental and physical effects, such as a “high” or “stoned” feeling, a general change in perception, euphoria, and an increase in appetite. Many individuals use marijuana in modern society for medical purposes. Unique means for intaking marijuana is desirable.

U.S. Pub. No. 2012/0124704 to Lucas Szymanowski relates to the infusion of cannabis. The described infusion of cannabis includes a method for infusing Cannabis. The method includes preparing the Cannabis. The method also includes placing the Cannabis in an infusible liquid. The infusible liquid is suitable for human consumption and configured to remove one or more chemical compounds from the infusible liquid.

### SUMMARY OF THE INVENTION

In view of the foregoing disadvantages inherent in the known beverage solution art, the present disclosure provides a novel marijuana beverage spiker. The general purpose of the present disclosure, which will be described subsequently in greater detail, is to provide an infusible liquid suitable for human consumption.

A marijuana beverage spiker system is disclosed herein comprising: a volume of liquid beverage, a beverage container, a volume of marijuana, and a mesh bag; wherein the marijuana beverage spiker system comprises the beverage container removably containing the volume of liquid beverage and the mesh bag containing the volume of marijuana. The mesh bag containing the volume of marijuana is immersed into the volume of liquid beverage held in the beverage container, with cannabinoids of the volume of marijuana being absorbed by the volume of liquid beverage thereby creating a psycho-active-solution (infused liquid) for human consumption.

According to another embodiment, a marijuana beverage spiker system is also disclosed herein. The marijuana beverage spiker system includes a method of making a marijuana beverage spiker beverage comprising the steps of: providing the marijuana beverage spiker system, opening a beverage container, inserting and immersing a (food grade) mesh bag filled with a volume of marijuana into a volume of liquid beverage within the beverage container, and allowing cannabinoids of the volume of marijuana to be absorbed by the volume of liquid beverage to create a psycho-active-solution for consumption. The method may further comprise the step of drinking the psycho-active-solution.

It should be noted that if decarboxylated marijuana is used, decarboxylation of the marijuana must occur first, wherein the marijuana is heated (without changing the shelf-life). The infusion takes a short period of time (i.e. minutes) at room temperature. Flavor may be picked up from the marijuana, but existing flavored drinks can still be made since the infused flavor isn't over-powering.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a marijuana beverage spiker, constructed and operative according to the teachings of the present disclosure.

FIG. 1 is a perspective view of the marijuana beverage spiker system during an ‘in-use’ condition, according to an embodiment of the disclosure.

FIG. 2 is a perspective view of the marijuana beverage spiker of FIG. 1, according to an embodiment of the present disclosure.

FIG. 3 is a perspective view of the marijuana beverage spiker of FIG. 1, according to an embodiment of the present disclosure.

FIG. 4 is a perspective view of the marijuana beverage spiker of FIG. 1, according to an embodiment of the present disclosure.

FIG. 5 is a flow diagram illustrating a method of making a marijuana beverage spiker beverage, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to beverage solutions and more particularly to a marijuana beverage spiker as used to improve the ingestion of marijuana.

Generally, the present invention is a mesh bag filled with decarboxylated marijuana, non-decarboxylated marijuana and/or marijuana concentrates designed to fit in virtually any beverage container. Once submerged the liquid in the beverage container absorbs the cannabinoids (THC/CBDs) and thereby becomes psycho-active. The Marijuana Beverage spiker is a food grade mesh bag filled with non-decarboxylated marijuana, decarboxylated marijuana and/or marijuana concentrates. The mesh bag is designed to fit into virtually any beverage container and is to be added to the container by the consumer after purchase. Once submerged in liquid, the water-soluble marijuana and/or marijuana concentrates in the spiker absorb into the liquid. The liquid will absorb the cannabinoids (THC/CBDs) and become psycho-active. The consumer can then feel the effects of the cannabinoids that are absorbed into the beverage much like a traditional ‘body stone’ (similar to other edible marijuana products).

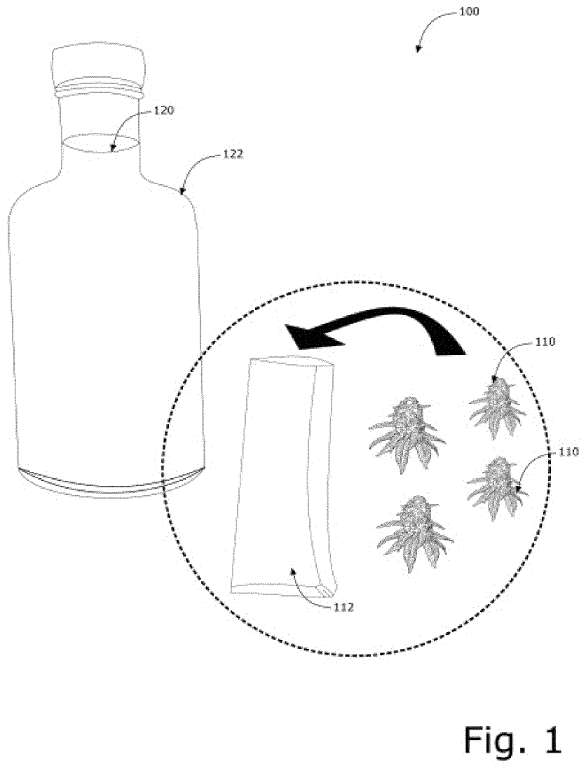

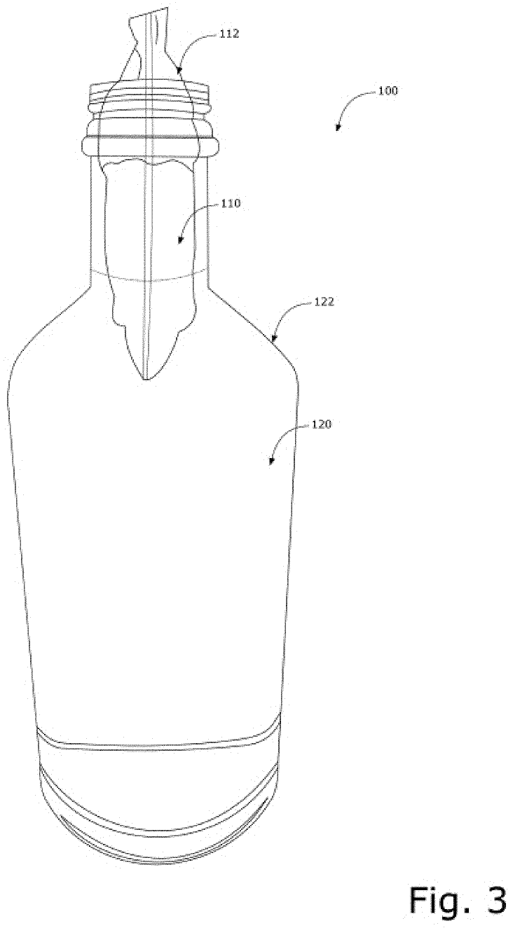

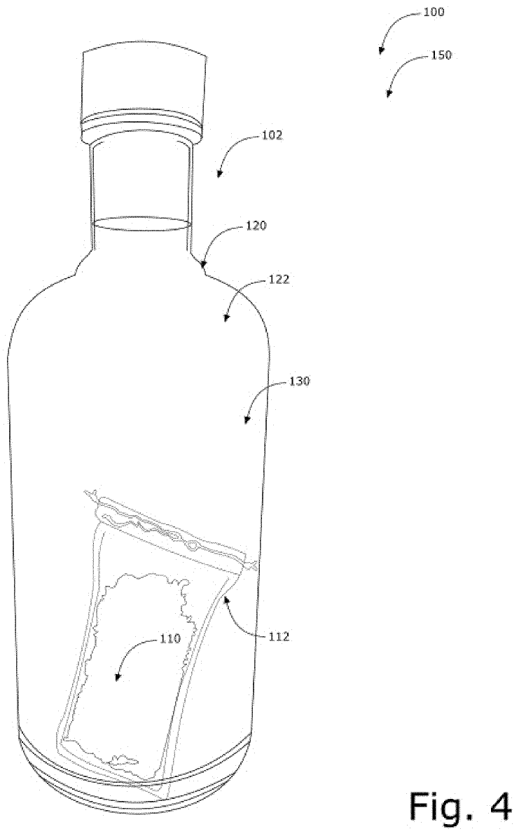

Referring now more specifically to the drawings by numerals of reference, there is shown in FIGS. 1-4, various views of a marijuana beverage spiker system 100.

FIG. 1 shows a marijuana beverage spiker system 100 during an ‘in-use’ condition 50, according to an embodiment of the present disclosure. Here, the marijuana beverage spiker system 100 may be beneficial for use by a consumer to flavorably ingest marijuana 110. As illustrated, the marijuana beverage spiker system 100 may include a volume of liquid beverage 120, a beverage container 122, a volume of marijuana 110, and a mesh bag 112; wherein the marijuana beverage spiker system 100 comprises the beverage container 122 removably containing the volume of liquid beverage 120 and the mesh bag 112 containing the volume of marijuana 110. The mesh bag 112 containing the volume of marijuana 110 is immersed into the volume of liquid beverage 120 held in the beverage container 122, cannabinoids and water-soluble elements of the volume of marijuana 110 being absorbed by the volume of liquid beverage 120 thereby creating a psycho-active-solution 130 (infused liquid) for human consumption.

According to one embodiment, the marijuana beverage spiker system 100 may be arranged as a kit. In particular, the marijuana beverage spiker system 100 may further include a set of instructions. The instructions may detail functional relationships in relation to the structure of the marijuana beverage spiker system 100 such that the marijuana beverage spiker 102 can be mixed, consumed, stored, or the like, in a preferred manner. Various strains of marijuana 110 and various types of liquid beverage 120 may be used and included in the kit combined with the other components mentioned herein. The preferred beverage container 122 used with the marijuana beverage spiker system 100 is a bottle with a removable lid, as shown however it should be appreciated that other container means may be used such as a can or the like. The volume of liquid beverage 120 may comprise water, fruit juice, vegetable juice, carbonated water or soda, sports drinks, energy drinks, caffeinated beverages (teas, coffees) and combinations thereof. The volume of liquid beverage generally comprises water. The above-mentioned strains of marijuana and liquids are intended to be exemplary and are not to be considered an exhaustive list instead are provided such that the mentioned invention can be enabled for making and consuming. Those with ordinary skill in the art will now appreciate that upon reading this specification and by their understanding the art of strains of marijuana and types of liquid beverage and their mixing in combination as described herein, methods of aging, mixing and consuming will be understood by those knowledgeable in such art.

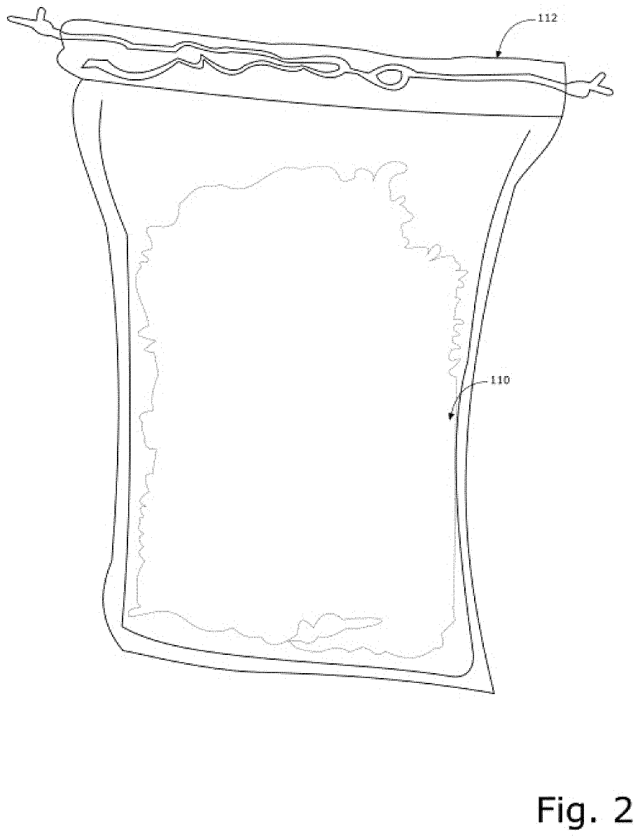

FIGS. 2-4 show various views of components of the marijuana beverage spiker system 100 of FIG. 1, according to an embodiment of the present disclosure. As above, the marijuana beverage spiker system 100 may include the mesh bag 112 comprises a food-grade-mesh-bag such that the psycho-active-solution 130 produced is suitable for human consumption by individuals of age and where law permits. The cannabinoids may comprise THC (Tetrahydrocannabinol) and/or CBDs (Cannabidiol); wherein the volume of marijuana comprises non-decarboxylated marijuana, decarboxylated-marijuana, or a combination thereof. The volume of marijuana 110 may also comprise marijuana-concentrates or combinations thereof.

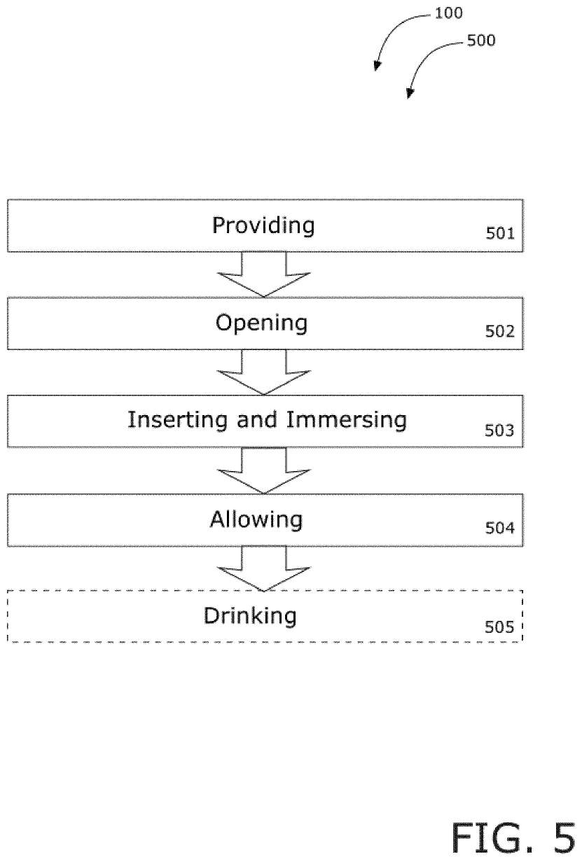

Referring now to FIG. 5 showing a flow diagram illustrating a method of making 500 a marijuana beverage spiker 102 beverage, according to an embodiment of the present disclosure. In particular, the method for making 500 a marijuana beverage spiker 102 beverage may include one or more components or features of the marijuana beverage spiker system 100 as described above. As illustrated, the method for making 500 may include the steps of: step one 501, providing the marijuana beverage spiker system; step two 502, opening a beverage container; step three 503, inserting and immersing a (food grade) mesh bag filled with a volume of marijuana into a volume of liquid beverage within the beverage container; step four 504 allowing cannabinoids of the volume of marijuana to be absorbed by the volume of liquid beverage to create a psycho-active-solution for consumption. The method 500 may further comprise the step five 505 of drinking the psycho-active-solution.

It should be noted that step 505 is an optional step and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for making, storing, mixing, using in recipes are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A marijuana beverage spiker system comprising:
a volume of a liquid beverage;
a beverage container;
a volume of marijuana; and
a mesh bag;
wherein said marijuana beverage spiker system comprises said beverage container removably containing said volume of liquid beverage and said mesh bag containing said volume of marijuana; and
wherein said mesh bag containing said volume of marijuana is immersed into said volume of liquid beverage held in said beverage container, cannabinoids of said volume of marijuana being absorbed by said volume of liquid beverage thereby creating a psycho-active-solution for consumption.

2. The marijuana beverage spiker system of claim 1, wherein the mesh bag comprises a food-grade-mesh-bag.

3. The marijuana beverage spiker system of claim 1, wherein said cannabinoids comprise THC.

4. The marijuana beverage spiker system of claim 1, wherein said cannabinoids comprise CBDs.

5. The marijuana beverage spiker system of claim 1, wherein the volume of marijuana comprises one or more of: non-decarboxylated marijuana and decarboxylated-marijuana.

6. The marijuana beverage spiker system of claim 1, wherein the volume of marijuana comprises marijuana-concentrates.

7. The marijuana beverage spiker system of claim 1, wherein the beverage container is a bottle with a removable lid.

8. The marijuana beverage spiker system of claim 1, wherein the beverage container is a can.

9. The marijuana beverage spiker system of claim 1, wherein the volume of liquid beverage comprises water.

10. The marijuana beverage spiker system of claim 1, wherein the volume of liquid beverage comprises carbonated soda.

11. The marijuana beverage spiker system of claim 1, wherein the volume of liquid beverage comprises fruit juice.

12. The marijuana beverage spiker system of claim 1, wherein the volume of liquid beverage comprises vegetable juice.

13. The marijuana beverage spiker system of claim 1, wherein the volume of liquid beverage comprises flavored water.

14. The marijuana beverage spiker system of claim 1, wherein the volume of liquid beverage comprises an energy drink.

15. The marijuana beverage spiker system of claim 1, wherein the volume of liquid beverage comprises a sports drink.

16. The marijuana beverage spiker system of claim 1, wherein the volume of liquid beverage comprises a caffeinated beverage.

17. A marijuana beverage spiker system comprising:
a volume of liquid beverage;
a beverage container;
a volume of marijuana; and
a mesh bag;
wherein said marijuana beverage spiker system comprises said beverage container removably containing said volume of liquid beverage and said mesh bag containing said volume of marijuana;
wherein the mesh bag comprises a food-grade-mesh-bag;
wherein the volume of marijuana comprises one or more of: non-decarboxylated marijuana, decarboxylated-marijuana and marijuana-concentrates;
wherein the beverage container is a bottle with a removable lid;
wherein the volume of liquid beverage comprises water; and
wherein said mesh bag containing said volume of marijuana is immersed into said volume of liquid beverage held in said beverage container, cannabinoids of said volume of marijuana being absorbed by said volume of liquid beverage thereby creating a psycho-active-solution for consumption.

18. The marijuana beverage spiker system of claim 17, further comprising set of instructions; and
wherein the marijuana beverage spiker system is arranged as a kit.

19. A method of making a marijuana beverage spiker system, the method comprising the steps of:
providing a marijuana beverage spiker system;
opening a beverage container;
inserting and immersing a mesh bag filled with a volume of marijuana into a volume of liquid beverage within said beverage container; and
allowing cannabinoids of said volume of marijuana to be absorbed by said volume of liquid beverage to create a psycho-active-solution for consumption.

20. The method of claim 19, further comprising the step of drinking the psycho-active-solution.
